(19) United States
(12) Patent Application Publication
(10) Pub. No.: US 2022/0246276 A1
(43) Pub. Date: Aug. 4, 2022

(54) SYSTEMS AND METHODS FOR GENERATING A NUTRITIVE PLAN TO MANAGE A UROLOGICAL DISORDER

(71) Applicant: KPN INNOVATIONS, LLC., Lakewood, CO (US)

(72) Inventor: Kenneth Neumann, LAKEWOOD, CO (US)

(73) Assignee: KPN INNOVATIONS, LLC., LAKEWOOD, CO (US)

(21) Appl. No.: 17/164,602

(22) Filed: Feb. 1, 2021

(51) Int. Cl. G16H 20/60; A61B 5/00; A61B 5/20; G16H 50/30

(52) U.S. Cl. CPC G16H 20/60; A61B 5/7267; G16H 50/30; A61B 5/4836; A61B 5/20

(57) ABSTRACT

A system for generating a nutritive plan to manage a urological disorder is disclosed. The system comprises a computing device configured to receive an input which includes physiological data. The computing device extracts at least one disease marker related to at least one urological disorder. A disease marker classifier is generated by the computing device. The disease marker classifier is generated by receiving disease marker training data correlating disease markers related to urological disorders to a urological disorder label. The disease marker classifier is trained using the disease marker training data. The disease marker classifier is used to classify the at least disease marker to a urological disorder label. A nutritive plan is generated as a function of the urological disorder label.

### FIELD OF THE INVENTION

The present invention relates to the field of nutrition for disease management. In particular, the present invention is directed to a system and method for generating a nutritive plan to manage urological disorders.

### BACKGROUND

Nutrition is an essential function of life as it provides the necessary nutrients the body needs to sustain all functions of life. The use of artificial intelligence in the field of nutrition may assist in the development and management of a healthy lifestyle for an individual.

### SUMMARY OF THE DISCLOSURE

In an aspect of the disclosure, a system for generating a nutritive plan to manage a urological disorder is disclosed. The system comprises a computing device configured to receive an input which includes physiological data. The computing device extracts at least one disease marker related to at least one urological disorder. A disease marker classifier is generated by the computing device. The disease marker classifier is generated by receiving disease marker training data correlating disease markers related to urological disorders to a urological disorder label. The disease marker classifier is trained using the disease marker training data. The disease marker classifier is used to classify the at least disease marker to a urological disorder label. A nutritive plan is generated as a function of the urological disorder label.

In another aspect of the disclosure, a method for generating a nutritive plan to manage a urological disorder is disclosed. The method includes receiving an input, by a computing device, which includes physiological data. At least one disease marker is extracted by the computing device, where the disease marker is related to at least one urological disorder. The computing device then generates a disease marker classifier. The disease marker classifier is generated by receiving disease marker training data correlating disease markers related to urological disorders to a urological disorder label. Computing device trains the disease marker classifier using the disease marker training data. The disease marker classifier is used to classify the at least disease marker to a urological disorder label. A nutritive plan is generated as a function of the urological disorder label.

These and other aspects and features of non-limiting embodiments of the present invention will become apparent to those skilled in the art upon review of the following description of specific non-limiting embodiments of the invention in conjunction with the accompanying drawings.

### BRIEF DESCRIPTION OF THE DRAWINGS

For the purpose of illustrating the invention, the drawings show aspects of one or more embodiments of the invention. However, it should be understood that the present invention is not limited to the precise arrangements and instrumentalities shown in the drawings, wherein:

FIG. 1 is a block diagram of an exemplary embodiment of a system of a nutritive plan to manage a urological disorder;

FIG. 2 is a block diagram of an exemplary embodiment of a database;

FIG. 3 is a block diagram of an exemplary embodiment of a machine-learning module;

FIG. 4. is a representative illustration of a nutritive plan in a GUI-based device;

FIG. 5 is a block diagram of an exemplary embodiment of a determination of a nutritive plan as a function of a machine-learning process;

FIG. 6 is a flow diagram illustrating an exemplary embodiment of a method of determining a nutritive plan to manage a urological condition; and

FIG. 7 is a block diagram of a computing system that can be used to implement any one or more of the methodologies disclosed herein and any one or more portions thereof.

The drawings are not necessarily to scale and may be illustrated by phantom lines, diagrammatic representations and fragmentary views. In certain instances, details that are not necessary for an understanding of the embodiments or that render other details difficult to perceive may have been omitted.

### DETAILED DESCRIPTION

At a high level, aspects of the present disclosure are directed to systems and methods for generating a nutritive plan to manage urological disorders. The system may include a computing device that may receive an input including physiological data which may be in the form of body fluids which include, but not limited to urine, semen, blood, and the like. A disease marker that may correspond to a urological disorder is extracted. A trained machine-learning classifier may be used to classify at least one disease marker that may correspond to a urological disorder to a urological label. Disease markers indicating a potential urological disorder may be extracted from a research journal or experimentally determined. Based on a disease marker related to a urological disorder, a nutritive plan is generated.

A practical application of this technology includes the use of a machine-learning process to provide a user access to nutritive plans that may improve and/or relieve symptoms related to urological disorder. The systems and methods allow for an update of the comestible plan if the urological disorder does not improve.

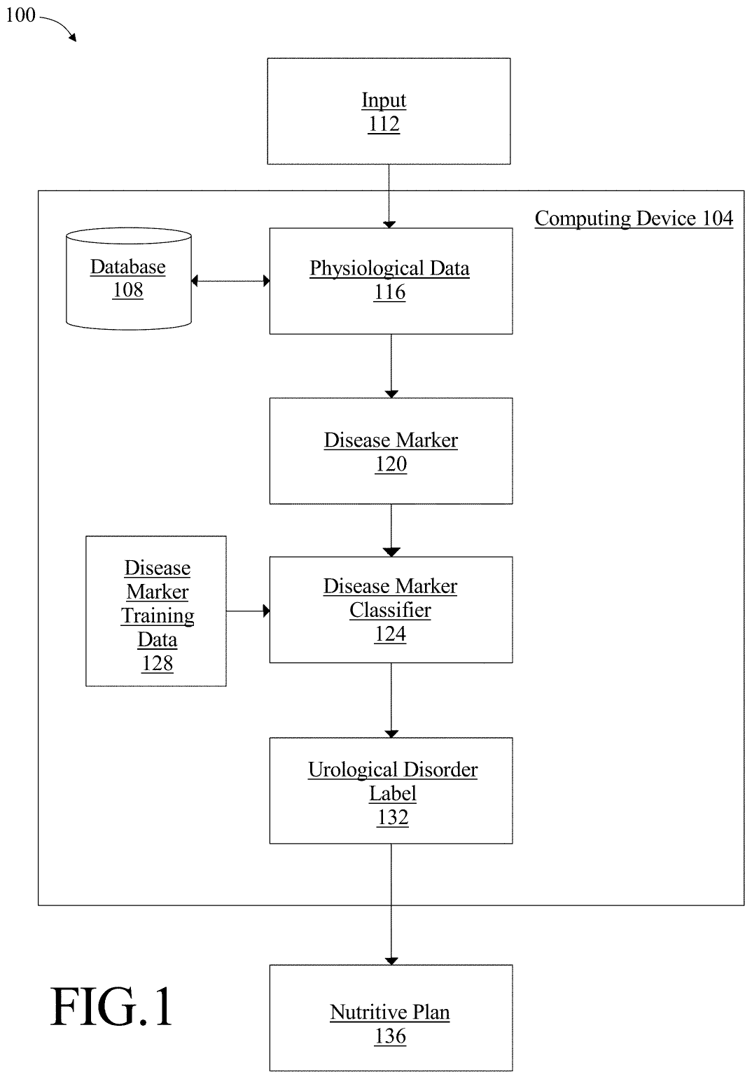

Referring now to FIG. 1, an exemplary embodiment of a system 100 for generating a nutritive plan to manage urological disorders is illustrated. As used in this disclosure, “manage” includes the prevention, treatment, and post-disorder maintenance with a nutritive plan of any urological disorder. System includes a computing device 104. Computing device 104 may include any computing device as described in this disclosure, including without limitation a microcontroller, microprocessor, digital signal processor (DSP) and/or system on a chip (SoC) as described in this disclosure. Computing device 104 may include, be included in, and/or communicate with a mobile device such as a mobile telephone or smartphone. Computing device 104 may include a single computing device operating independently, or may include two or more computing device operating in concert, in parallel, sequentially or the like; two or more computing devices may be included together in a single computing device or in two or more computing devices. Computing device 104 may interface or communicate with one or more additional devices as described below in further detail via a network interface device. Network interface device may be utilized for connecting computing device 104 to one or more of a variety of networks, and one or more devices. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus or other relatively small geographic space), a telephone network, a data network associated with a telephone/voice provider (e.g., a mobile communications provider data and/or voice network), a direct connection between two computing devices, and any combinations thereof. A network may employ a wired and/or a wireless mode of communication. In general, any network topology may be used. Information (e.g., data, software etc.) may be communicated to and/or from a computer and/or a computing device. Computing device 104 may include but is not limited to, for example, a computing device or cluster of computing devices in a first location and a second computing device or cluster of computing devices in a second location. Computing device 104 may include one or more computing devices dedicated to data storage, security, distribution of traffic for load balancing, and the like. Computing device 104 may distribute one or more computing tasks as described below across a plurality of computing devices of computing device, which may operate in parallel, in series, redundantly, or in any other manner used for distribution of tasks or memory between computing devices. Computing device 104 may be implemented using a “shared nothing” architecture in which data is cached at the worker, in an embodiment, this may enable scalability of system 100 and/or computing device.

With continued reference to FIG. 1, computing device 104 may be designed and/or configured to perform any method, method step, or sequence of method steps in any embodiment described in this disclosure, in any order and with any degree of repetition. For instance, computing device 104 may be configured to perform a single step or sequence repeatedly until a desired or commanded outcome is achieved; repetition of a step or a sequence of steps may be performed iteratively and/or recursively using outputs of previous repetitions as inputs to subsequent repetitions, aggregating inputs and/or outputs of repetitions to produce an aggregate result, reduction or decrement of one or more variables such as global variables, and/or division of a larger processing task into a set of iteratively addressed smaller processing tasks. Computing device 104 may perform any step or sequence of steps as described in this disclosure in parallel, such as simultaneously and/or substantially simultaneously performing a step two or more times using two or more parallel threads, processor cores, or the like; division of tasks between parallel threads and/or processes may be performed according to any protocol suitable for division of tasks between iterations. Persons skilled in the art, upon reviewing the entirety of this disclosure, will be aware of various ways in which steps, sequences of steps, processing tasks, and/or data may be subdivided, shared, or otherwise dealt with using iteration, recursion, and/or parallel processing.

Still referring to FIG. 1, computing device 104 may connect to and/or include a database 108. Database 108 may be implemented, without limitation, as a relational database 108, a key-value retrieval database 108 such as a NOSQL database 108, or any other format or structure for use as a database 108 that a person skilled in the art would recognize as suitable upon review of the entirety of this disclosure. Database 108 may alternatively or additionally be implemented using a distributed data storage protocol and/or data structure, such as a distributed hash table or the like. Database 108 may include a plurality of data entries and/or records as described above. Data entries in a database 108 may be flagged with or linked to one or more additional elements of information, which may be reflected in data entry cells and/or in linked tables such as tables related by one or more indices in a relational database 108. Persons skilled in the art, upon reviewing the entirety of this disclosure, will be aware of various ways in which data entries in a database 108 may store, retrieve, organize, and/or reflect data and/or records as used herein, as well as categories and/or populations of data consistently with this disclosure. In some embodiments, network data, or other information such as user information, transfer party information, and alimentary provider information, may be stored in and/or retrieved from database 108.

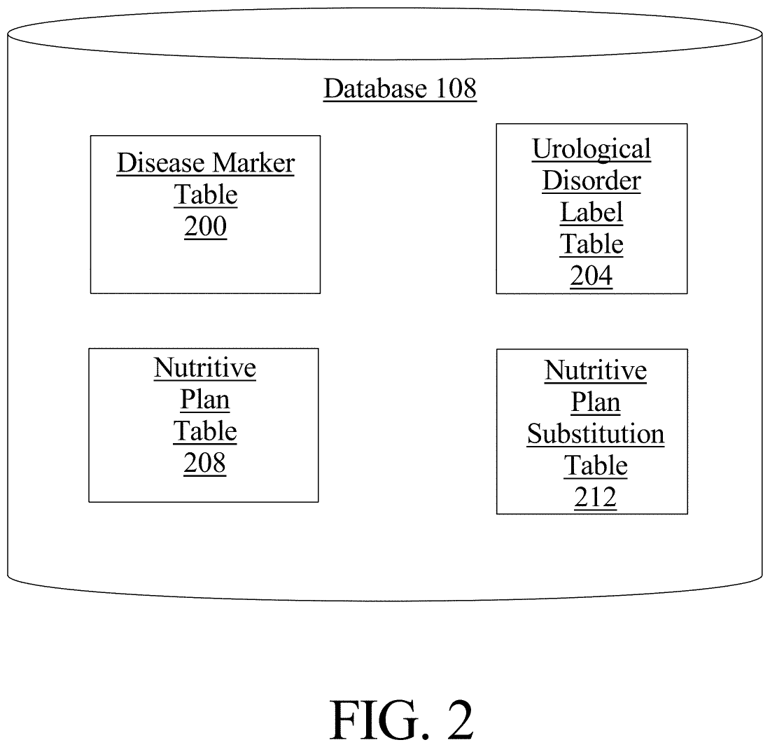

Referring now to FIG. 2 an exemplary embodiment of a database 108 is illustrated. Database 108 may, as a non-limiting example, organize data stored in the database according to one or more database tables. One or more database tables may be linked to one another by, for instance, common column values. For instance, a common column between two tables of database 108 may include an identifier of alimentary providers, for instance as defined below; as a result, a query may be able to retrieve all rows from any table pertaining to a given alimentary provider. Other columns may include any other category usable for organization or subdivision of data, including types of data, common pathways between, for example, an alimentary combination and a first alimentary provider, or the like; persons skilled in the art, upon reviewing the entirety of this disclosure, will be aware of various ways in which expert data from one or more tables may be linked and/or related to expert data in one or more other tables.

Still referring to FIG. 2, one or more database tables in database 108 may include, as a non-limiting example, a disease marker table 200. Disease marker table 200 may be used to store disease markers corresponding to urological disorders, correlations between disease markers corresponding to urological disorder and other health disorders, or the like. As another non-limiting example, one or more tables in database 108 may include a urological disorder label table 204. Urological disorder label table 204 may be used to store correlations between disease markers and potential urological disorders, and the like. Another non-limiting example, one or more tables in database 108 may include a nutritive plan table 208. Nutritive plan table 208 may include, but not limited to nutritive combinations that may treat or prevent a specific urological disorder, adverse foods affecting urological disorders, and the like. As another non-limiting example, one or more tables in database 108 may include nutritive plan substitution table 212. Nutritive plan substitution table 212 may include alimentary combinations that may include allowable substitutions for alimentary combinations, substitutions that may create an adverse effect on a urological condition, and the like.

With continued reference to FIG. 1, computing device 104 may be configured to receive an input 112. An “input,” as used in this disclosure, may include, but not limited to any medical test, a user's health assessment, a user's nutritional assessment, an assessment conducted in any website related to a urological condition, a direct entry from a user, and the like. Input 112 may include physiological data 116. As used in this disclosure, “physiological data” is any data indicative of a person's physiological state; physiological state may be evaluated with regard to one or more measures of health of a person's body, one or more systems within a person's body such as a circulatory system, a digestive system, a nervous system, or the like, one or more organs within a person's body, and/or any other subdivision of a person's body useful for diagnostic or prognostic purposes. For instance, and without limitation, a particular set of biomarkers, test results, and/or biochemical information may be recognized in each medical field as useful for identifying various disease conditions or prognoses within a relevant field. As a non-limiting example, and without limitation, physiological data 116 which may include any abnormalities found from a digital rectal exam (DRE) or elevated levels of prostate-specific statin (PSA) may indicate a likelihood of prostate cancer in men.

Still with reference to FIG. 1, physiological data may include bodily fluids. A biological fluid, as defined in this disclosure, is any fluid produced by the human body. A biological fluid may be excreted, such as, for example but not limited to, urine or sweat. A biological fluid may be secreted such as, for example but not limited to, breast milk or bile. A biological fluid may be obtained with a needle such as, for example, but not limited to blood or cerebrospinal fluid. A biological fluid may develop as a result of a pathological process such as, for example but not limited to, blister or cyst fluid. Biological fluids include blood, urine, semen (seminal fluid), vaginal secretions, cerebrospinal fluid (CSF), synovial fluid, pleural fluid (pleural lavage), pericardial fluid, peritoneal fluid, amniotic fluid, saliva, nasal fluid, optic fluid, gastric fluid, breast milk, as well as cell culture supernatants.

With continued reference to FIG. 1, physiological data may include, without limitation, hematological data, such as red blood cell count, which may include a total number of red blood cells in a person's blood and/or in a blood sample, hemoglobin levels, hematocrit representing a percentage of blood in a person and/or sample that is composed of red blood cells, mean corpuscular volume, which may be an estimate of the average red blood cell size, mean corpuscular hemoglobin, which may measure average weight of hemoglobin per red blood cell, mean corpuscular hemoglobin concentration, which may measure an average concentration of hemoglobin in red blood cells, platelet count, mean platelet volume which may measure the average size of platelets, red blood cell distribution width, which measures variation in red blood cell size, absolute neutrophils, which measures the number of neutrophil white blood cells, absolute quantities of lymphocytes such as B-cells, T-cells, Natural Killer Cells, and the like, absolute numbers of monocytes including macrophage precursors, absolute numbers of eosinophils, and/or absolute counts of basophils. Physiological data 116 may include, without limitation, immune function data such as Interleukine-6 (IL-6), TNF-alpha, systemic inflammatory cytokines, and the like.

Continuing to refer to FIG. 1, physiological data 116 may include, without limitation, data describing blood-born lipids, including total cholesterol levels, high-density lipoprotein (HDL) cholesterol levels, low-density lipoprotein (LDL) cholesterol levels, very low-density lipoprotein (VLDL) cholesterol levels, levels of triglycerides, and/or any other quantity of any blood-born lipid or lipid-containing substance. Physiological data 116 may include measures of glucose metabolism such as fasting glucose levels and/or hemoglobin A1-C(HbA1c) levels. Physiological data 116 may include, without limitation, one or more measures associated with endocrine function, such as without limitation, quantities of dehydroepiandrosterone (DHEAS), DHEA-Sulfate, quantities of cortisol, ratio of DHEAS to cortisol, quantities of testosterone quantities of estrogen, quantities of growth hormone (GH), insulin-like growth factor 1 (IGF-1), quantities of adipokines such as adiponectin, leptin, and/or ghrelin, quantities of somatostatin, progesterone, or the like. Physiological data 116 may include measures of estimated glomerular filtration rate (eGFR). Physiological data 116 may include quantities of C-reactive protein, estradiol, ferritin, folate, homocysteine, prostate-specific Ag, thyroid-stimulating hormone, vitamin D, 25 hydroxy, blood urea nitrogen, creatinine, sodium, potassium, chloride, carbon dioxide, uric acid, albumin, globulin, calcium, phosphorus, alkaline phosphatase, alanine amino transferase, aspartate amino transferase, lactate dehydrogenase (LDH), bilirubin, gamma-glutamyl transferase (GGT), iron, and/or total iron binding capacity (TIBC), or the like. Physiological data 116 may include antinuclear antibody levels. Physiological data 116 may include aluminum levels. Physiological data 116 may include arsenic levels. Physiological data 116 may include levels of fibrinogen, plasma cystatin C, and/or brain natriuretic peptide.

Continuing to refer to FIG. 1, physiological data 116 may include measures of lung function such as forced expiratory volume, one second (FEV-1) which measures how much air can be exhaled in one second following a deep inhalation, forced vital capacity (FVC), which measures the volume of air that may be contained in the lungs. Physiological data 116 may include a measurement blood pressure, including without limitation systolic and diastolic blood pressure. Physiological data 116 may include a measure of waist circumference. Physiological data 116 may include body mass index (BMI). Physiological data 116 may include one or more measures of bone mass and/or density such as dual-energy x-ray absorptiometry. Physiological data 116 may include one or more measures of muscle mass. Physiological data 116 may include one or more measures of physical capability such as without limitation measures of grip strength, evaluations of standing balance, evaluations of gait speed, pegboard tests, timed up and go tests, and/or chair rising tests.

Still viewing FIG. 1, physiological data 116 may include one or more measures of cognitive function, including without limitation Rey auditory verbal learning test results, California verbal learning test results, NIH toolbox picture sequence memory test, Digital symbol coding evaluations, and/or Verbal fluency evaluations. Physiological data 116 may include one or more evaluations of sensory ability, including measures of audition, vision, olfaction, gustation, vestibular function and pain.

Continuing to refer to FIG. 1, physiological data 116 may include psychological data. Psychological data may include any data generated using psychological, neuro-psychological, and/or cognitive evaluations, as well as diagnostic screening tests, personality tests, personal compatibility tests, or the like; such data may include, without limitation, numerical score data entered by an evaluating professional and/or by a subject performing a self-test such as a computerized questionnaire. Psychological data may include textual, video, or image data describing testing, analysis, and/or conclusions entered by a medical professional such as without limitation a psychologist, psychiatrist, psychotherapist, social worker, a medical doctor, or the like. Psychological data may include data gathered from user interactions with persons, documents, and/or computing devices; for instance, user patterns of purchases, including electronic purchases, communication such as via chatrooms or the like, any textual, image, video, and/or data produced by the subject, any textual image, video and/or other data depicting and/or describing the subject, or the like. Any psychological data and/or data used to generate psychological data may be analyzed using machine-learning and/or language processing modules as described in this disclosure.

Still referring to FIG. 1, physiological data 116 may include genomic data, including deoxyribonucleic acid (DNA) samples and/or sequences, such as without limitation DNA sequences contained in one or more chromosomes in human cells. Genomic data may include, without limitation, ribonucleic acid (RNA) samples and/or sequences, such as samples and/or sequences of messenger RNA (mRNA) or the like taken from human cells. Genetic data may include telomere lengths. Genomic data may include epigenetic data including data describing one or more states of methylation of genetic material. Physiological data 116 may include proteomic data, which as used herein is data describing all proteins produced and/or modified by an organism, colony of organisms, or system of organisms, and/or a subset thereof. Physiological data 116 may include data concerning a microbiome of a person, which as used herein includes any data describing any microorganism and/or combination of microorganisms living on or within a person, including without limitation biomarkers, genomic data, proteomic data, and/or any other metabolic or biochemical data useful for analysis of the effect of such microorganisms on other physiological data 116 of a person, and/or on prognostic labels and/or ameliorative processes as described in further detail below.

With continuing reference to FIG. 1, physiological data 116 may include one or more user-entered descriptions of a person's physiological state. One or more user-entered descriptions may include, without limitation, user descriptions of symptoms, which may include without limitation current or past physical, psychological, perceptual, and/or neurological symptoms, user descriptions of current or past physical, emotional, and/or psychological problems and/or concerns, user descriptions of past or current treatments, including therapies, nutritional regimens, exercise regimens, pharmaceuticals or the like, or any other user-entered data that a user may provide to a medical professional when seeking treatment and/or evaluation, and/or in response to medical intake papers, questionnaires, questions from medical professionals, or the like. Physiological data 116 may include any physiological data 116, as described above, describing any multicellular organism living in or on a person including any parasitic and/or symbiotic organisms living in or on the persons; non-limiting examples may include mites, nematodes, flatworms, or the like. Examples of physiological data 116 described in this disclosure are presented for illustrative purposes only and are not meant to be exhaustive.

With continued reference to FIG. 1, physiological data 116 may include, without limitation any result of any medical test, physiological assessment, cognitive assessment, psychological assessment, or the like. For instance, physiological data 116 may include any medical tests and/or results used to diagnose a urological disorder. As used in this specification, a “urological disorder” includes any diseases, disorders or conditions that affect the kidneys, ureters, bladder, prostate in men, or urethra, or that affect their function. Examples of urinary disorders may include, but not limited to of the urinary tract, incontinence (inability to control urine flow), interstitial cystitis, kidney stones, kidney failure, or urinary tract infections. Urological disorders may be caused, for instance, by cancer, conditions affecting the structures near the urinary tract, infection, inflammation, injury, nervous system diseases, scarring, or urine crystallization. A non-limiting example of a medical test used in the determination of a urological disorder may include a urinalysis. A urinalysis is a test of urine. A urinalysis is used to detect and/or manage a wide range of disorders, such as, but not limited to urinary tract infections, kidney disease and diabetes. A urinalysis involves checking the appearance, concentration and content of urine. Another non-limiting example of a test to detect a urological disorder may be a prostate-specific antigen test. The test measures the amount of prostate-specific antigen (PSA) in your blood. A “PSA”, as used in this disclosure, is a protein produced by both cancerous and noncancerous tissue in the prostate, a small gland that sits below the bladder in men. The PSA test may detect high levels of PSA that may indicate the presence of prostate cancer. Prostate-specific antigen (PSA) may include a glycoprotein enzyme encoded in humans by the KLK3 gene. PSA is a member of the kallikrein-related peptidase family and is secreted by the epithelial cells of the prostate gland. PSA is present in small quantities in the serum of men with healthy prostates, but is often elevated in the presence of prostate cancer or other prostate disorders. Physiological data 116 that includes results of a PSA test may help diagnose prostate cancer in men.

With continued reference to FIG. 1, input 112 may include at least a physiological data 116 from one or more other devices after performance; system 100 may alternatively or additionally perform one or more assessments and/or tests to obtain at least a physiological data 116, and/or one or more portions thereof, on system 100. For instance, at least physiological data 116 may include or more entries by a user in a form or similar graphical user interface object; one or more entries may include, without limitation, user responses to questions on a psychological, behavioral, personality, or cognitive test. For instance, at least a computing device 104 may present to user a set of assessment questions designed or intended to evaluate a current state of mind of the user, a current psychological state of the user, a personality trait of the user, or the like; at least a computing device 104 may provide user-entered responses to such questions directly as at least a physiological data 116 and/or may perform one or more calculations or other algorithms to derive a score or other result of an assessment as specified by one or more testing protocols, such as automated calculation of a Stanford-Binet and/or Wechsler scale for IQ testing, a personality test scoring such as a Myers-Briggs test protocol, or other assessments that may occur to persons skilled in the art upon reviewing the entirety of this disclosure.

With continued reference to FIG. 1, assessment and/or self-assessment data, and/or automated or other assessment results, obtained from a third-party device; third-party device may include, without limitation, a server or other device (not shown) that performs automated cognitive, psychological, behavioral, personality, or other assessments. Third-party device may include a device operated by an informed advisor. An informed advisor may include any medical professional who may assist and/or participate in the medical treatment of a user. An informed advisor may include a medical doctor, nurse, physician assistant, pharmacist, yoga instructor, nutritionist, spiritual healer, meditation teacher, fitness coach, health coach, life coach, and the like.

With continued reference to FIG. 1, physiological data 116 may include data describing one or more test results, including results of mobility tests, stress tests, dexterity tests, endocrinal tests, genetic tests, and/or electromyographic tests, biopsies, radiological tests, genetic tests, and/or sensory tests. Persons skilled in the art, upon reviewing the entirety of this disclosure, will be aware of various additional examples of at least a physiological sample consistent with this disclosure.

With continued reference to FIG. 1, physiological data 116 may include one or more user body measurements. A “user body measurement” as used in this disclosure, includes a measurable indicator of the severity, absence, and/or presence of a disease state. A “disease state” as used in this disclosure, includes any harmful deviation from the normal structural and/or function state of a human being. A disease state may include any medical condition and may be associated with specific symptoms and signs. A disease state may be classified into different types including infectious diseases, deficiency diseases, hereditary diseases, and/or physiological diseases. For instance and without limitation, internal dysfunction of the immune system may produce a variety of different diseases including immunodeficiency, hypersensitivity, allergies, and/or autoimmune disorders.

With continued reference to FIG. 1, user body measurements may be related to particular dimensions of the human body. A “dimension of the human body” as used in this disclosure, includes one or more functional body systems that are impaired by disease in a human body and/or animal body. Functional body systems may include one or more body systems recognized as attributing to root causes of disease by functional medicine practitioners and experts. A “root cause” as used in this disclosure, includes any chain of causation describing underlying reasons for a particular disease state and/or medical condition instead of focusing solely on symptomatology reversal. Root cause may include chains of causation developed by functional medicine practices that may focus on disease causation and reversal. For instance and without limitation, a medical condition such as diabetes may include a chain of causation that does not include solely impaired sugar metabolism but that also includes impaired hormone systems including insulin resistance, high cortisol, less than optimal thyroid production, and low sex hormones. Diabetes may include further chains of causation that include inflammation, poor diet, delayed food allergies, leaky gut, oxidative stress, damage to cell membranes, and dysbiosis. Dimensions of the human body may include but are not limited to epigenetics, gut-wall, microbiome, nutrients, genetics, and/or metabolism.

With continued reference to FIG. 1, epigenetic, as used herein, includes any user body measurements describing changes to a genome that do not involve corresponding changes in nucleotide sequence. Epigenetic body measurement may include data describing any heritable phenotypic. Phenotype, as used herein, include any observable trait of a user including morphology, physical form, and structure. Phenotype may include a user's biochemical and physiological properties, behavior, and products of behavior. Behavioral phenotypes may include cognitive, personality, and behavior patterns. This may include effects on cellular and physiological phenotypic traits that may occur due to external or environmental factors. For example, DNA methylation and histone modification may alter phenotypic expression of genes without altering underlying DNA sequence. Epigenetic body measurements may include data describing one or more states of methylation of genetic material.

With continued reference to FIG. 1, gut-wall, as used herein, includes the space surrounding the lumen of the gastrointestinal tract that is composed of four layers including the mucosa, submucosa, muscular layer, and serosa. The mucosa contains the gut epithelium that is composed of goblet cells that function to secrete mucus, which aids in lubricating the passage of food throughout the digestive tract. The goblet cells also aid in protecting the intestinal wall from destruction by digestive enzymes. The mucosa includes villi or folds of the mucosa located in the small intestine that increase the surface area of the intestine. The villi contain a lacteal, that is a vessel connected to the lymph system that aids in removal of lipids and tissue fluids. Villi may contain microvilli that increase the surface area over which absorption can take place. The large intestine lack villi and instead a flat surface containing goblet cells are present.

With continued reference to FIG. 1, gut-wall includes the submucosa, which contains nerves, blood vessels, and elastic fibers containing collagen. Elastic fibers contained within the submucosa aid in stretching the gastrointestinal tract with increased capacity while also maintaining the shape of the intestine. Gut-wall includes muscular layer which contains smooth muscle that aids in peristalsis and the movement of digested material out of and along the gut. Gut-wall includes the serosa which is composed of connective tissue and coated in mucus to prevent friction damage from the intestine rubbing against other tissue. Mesenteries are also found in the serosa and suspend the intestine in the abdominal cavity to stop it from being disturbed when a person is physically active.

With continued reference to FIG. 1, gut-wall body measurement may include data describing one or more test results including results of gut-wall function, gut-wall integrity, gut-wall strength, gut-wall absorption, gut-wall permeability, intestinal absorption, gut-wall barrier function, gut-wall absorption of bacteria, gut-wall malabsorption, gut-wall gastrointestinal imbalances and the like.

With continued reference to FIG. 1, gut-wall body measurement may include any data describing blood test results of creatinine levels, lactulose levels, zonulin levels, and mannitol levels. Gut-wall body measurement may include blood test results of specific gut-wall body measurements including d-lactate, endotoxin lipopolysaccharide (LPS) Gut-wall body measurement may include data breath tests measuring lactulose, hydrogen, methane, lactose, and the like. Gut-wall body measurement may include blood test results describing blood chemistry levels of albumin, bilirubin, complete blood count, electrolytes, minerals, sodium, potassium, calcium, glucose, blood clotting factors,

With continued reference to FIG. 1, gut-wall body measurement may include one or more stool test results describing presence or absence of parasites, firmicutes, Bacteroidetes, absorption, inflammation, food sensitivities. Stool test results may describe presence, absence, and/or measurement of acetate, aerobic bacterial cultures, anerobic bacterial cultures, fecal short chain fatty acids, beta-glucuronidase, cholesterol, chymotrypsin, fecal color, Cryptosporidium EIA, Entamoeba histolytica, fecal lactoferrin, Giardia lamblia EIA, long chain fatty acids, meat fibers and vegetable fibers, mucus, occult blood, parasite identification, phospholipids, propionate, putrefactive short chain fatty acids, total fecal fat, triglycerides, yeast culture, n-butyrate, pH and the like.

With continued reference to FIG. 1, gut-wall body measurement may include one or more stool test results describing presence, absence, and/or measurement of microorganisms including bacteria, archaea, fungi, protozoa, algae, viruses, parasites, worms, and the like. Stool test results may contain species such as Bifidobacterium species, Campylobacter species, Clostridium difficile, Cryptosporidium species, Cyclospora cayetanensis, Cryptosporidium EIA, Dientamoeba fragilis, Entamoeba histolytica, Escherichia coli, Entamoeba histolytica, Giardia, H. pylori, Candida albicans, Lactobacillus species, worms, macroscopic worms, mycology, protozoa, Shiga toxin E. coli, and the like.

With continued reference to FIG. 1, gut-wall body measurement may include one or more microscopic ova exam results, microscopic parasite exam results, protozoan polymerase chain reaction test results and the like. Gut-wall body measurement may include enzyme-linked immunosorbent assay (ELISA) test results describing immunoglobulin G (Ig G) food antibody results, immunoglobulin E (Ig E) food antibody results, Ig E mold results, IgG spice and herb results. Gut-wall body measurement may include measurements of calprotectin, eosinophil protein x (EPX), stool weight, pancreatic elastase, total urine volume, blood creatinine levels, blood lactulose levels, blood mannitol levels.

With continued reference to FIG. 1, gut-wall body measurement may include one or more elements of data describing one or more procedures examining gut including for example colonoscopy, endoscopy, large and small molecule challenge, and subsequent urinary recovery using large molecules such as lactulose, polyethylene glycol-3350, and small molecules such as mannitol, L-rhamnose, polyethyleneglycol-400. Gut-wall body measurement may include data describing one or more images such as x-ray, MRI, CT scan, ultrasound, standard barium follow-through examination, barium enema, barium with contract, MRI fluoroscopy, positron emission tomography 9PET), diffusion-weighted MRI imaging, and the like.

With continued reference to FIG. 1, microbiome, as used herein, includes ecological community of commensal, symbiotic, and pathogenic microorganisms that reside on or within any of a number of human tissues and biofluids. For example, human tissues and biofluids may include the skin, mammary glands, placenta, seminal fluid, uterus, vagina, ovarian follicles, lung, saliva, oral mucosa, conjunctiva, biliary, and gastrointestinal tracts. Microbiome may include for example, bacteria, archaea, protists, fungi, and viruses. Microbiome may include commensal organisms that exist within a human being without causing harm or disease. Microbiome may include organisms that are not harmful but rather harm the human when they produce toxic metabolites such as trimethylamine. Microbiome may include pathogenic organisms that cause host damage through virulence factors such as producing toxic by-products. Microbiome may include populations of microbes such as bacteria and yeasts that may inhabit the skin and mucosal surfaces in various parts of the body. Bacteria may include for example Firmicutes species, Bacteroidetes species, Proteobacteria species, Verrumicrobia species, Actinobacteria species, Fusobacteria species, Cyanobacteria species and the like. Archaea may include methanogens such as Methanobrevibacter smithies' and Methanosphaera stadtmanae. Fungi may include Candida species and Malassezia species. Viruses may include bacteriophages. Microbiome species may vary in different locations throughout the body. For example, the genitourinary system may contain a high prevalence of Lactobacillus species while the gastrointestinal tract may contain a high prevalence of Bifidobacterium species while the lung may contain a high prevalence of Streptococcus and Staphylococcus species.

With continued reference to FIG. 1, microbiome body measurement may include one or more stool test results describing presence, absence, and/or measurement of microorganisms including bacteria, archaea, fungi, protozoa, algae, viruses, parasites, worms, and the like. Stool test results may contain species such as Ackerman's muciniphila, Anaerotruncus colihominis, bacteriology, Bacteroides vulgates', Bacteroides-Prevotella, Barnesiella species, Bifidobacterium longarm, Bifidobacterium species, Butyrivbrio crossotus, Clostridium species, Collinsella aerofaciens, fecal color, fecal consistency, Coprococcus eutactus, Desulfovibrio piger, Escherichia coli, Faecalibacterium prausnitzii, Fecal occult blood, Firmicutes to Bacteroidetes ratio, Fusobacterium species, Lactobacillus species, Methanobrevibacter smithii, yeast minimum inhibitory concentration, bacteria minimum inhibitory concentration, yeast mycology, fungi mycology, Odoribacter species, Oxalobacter formigenes, parasitology, Prevotella species, Pseudoflavonifractor species, Roseburia species, Ruminococcus species, Veillonella species and the like.

With continued reference to FIG. 1, microbiome body measurement may include one or more stool tests results that identify all microorganisms living a user's gut including bacteria, viruses, archaea, yeast, fungi, parasites, and bacteriophages. Microbiome body measurement may include DNA and RNA sequences from live microorganisms that may impact a user's health. Microbiome body measurement may include high resolution of both species and strains of all microorganisms. Microbiome body measurement may include data describing current microbe activity. Microbiome body measurement may include expression of levels of active microbial gene functions. Microbiome body measurement may include descriptions of sources of disease-causing microorganisms, such as viruses found in the gastrointestinal tract such as raspberry bushy swarf virus from consuming contaminated raspberries or Pepino mosaic virus from consuming contaminated tomatoes.

With continued reference to FIG. 1, microbiome body measurement may include one or more blood test results that identify metabolites produced by microorganisms. Metabolites may include for example, indole-3-propionic acid, indole-3-lactic acid, indole-3-acetic acid, tryptophan, serotonin, kynurenine, total indoxyl sulfate, tyrosine, xanthine, 3-methylxanthine, uric acid, and the like.

With continued reference to FIG. 1, microbiome body measurement may include one or more breath test results that identify certain strains of microorganisms that may be present in certain areas of a user's body. This may include for example, lactose intolerance breath tests, methane-based breath tests, hydrogen-based breath tests, fructose-based breath tests. Helicobacter pylori breath test, fructose intolerance breath test, bacterial overgrowth syndrome breath tests and the like.

With continued reference to FIG. 1, microbiome body measurement may include one or more urinary analysis results for certain microbial strains present in urine. This may include for example, urinalysis that examines urine specific gravity, urine cytology, urine sodium, urine culture, urinary calcium, urinary hematuria, urinary glucose levels, urinary acidity, urinary protein, urinary nitrites, bilirubin, red blood cell urinalysis, and the like.

Still referring to FIG. 1, computing device 104 may extract at least one disease marker 120 related to at least one urological disorder. A “disease marker,” as used in this disclosure, is a biological element found in physiological data, for example, body fluids, that indicate the presence or absence of a condition or a disease. The presence of disease marker 120 may indicate a likelihood that a urological disorder at a future date. For instance, early detection of Meprin-1-alpha (MEP1a) in urine may indicate the potential for a user to develop acute renal failure at a future date. Disease marker 120 may include, for example, monitoring disease markers. A “monitoring disease marker,” as used in this specification, is a disease marker that may be used to assess the progress of a disease or to monitor the effects of a therapeutic agent, such as, for example, administration of a course of antibiotics. In another example, a disease marker may be a diagnostic disease marker. A “diagnostic disease marker,” as defined in this disclosure is a disease marker that is used to detect the presence of a disease or a condition of interest. In an embodiment, the plurality of disease markers comprises a diagnostic disease marker. Another example of a disease marker is a predictive disease marker. A “predictive disease marker,” as used in this disclosure, is a disease marker used to predict what group of patients will respond favorably or unfavorably to a particular treatment. Examples of disease marker 120 that may be used in diagnosing a urological disorder may include, but are not limited to SCUBE-1, sCD40L, aminopeptidase N, vasorin precursor, alpha-1-antitryptsin, ceruloplasmin, GFR, SCr, CysC, albuminuria, creatinine, MDRD, urea, uric acid, electrolytes, cystatin C, beta-trace protein, and the like. Disease marker 120 may be extracted, for example, chemically. For instance, an enzyme-linked immunosorbent assay (“ELISA”) may be used to identify at least one urologically-related disease marker. For instance, the presence of Interleukin IL-1β (IL-1β) and/or matrix metalloproteinase (MMP-9) may indicate the potential for the presence of a urological disorder. Disease marker 120 indicating a potential urological disorder may be extracted, for example, from a research journal. Alternatively, disease marker 120 may be extracted by experimentation. For example, a disease marker that may indicate a urological disorder may incorporate testing for the presence of disease marker 120 using a control group where there is no known urological disorder present. Values for the disease marker for a sample group known to have a urological disorder may be compared against the values obtained for the control group and a determination made regarding the presence of a urological disorder.

With continued reference to FIG. 1, computing device 104 may generate a disease marker classifier 124. Computing device 104 may receive disease marker training data 128 correlating disease markers related to urological disorders to urological disorder label 132. As used in this disclosure, “urological disorder labels” is an element of data denoting an identified and/or predicted urological disease based on the presence of a particular disease marker and/or the presence of a particular biomarker at a concentration above or below the normal concentration for that disease marker. For example, the presence of Tamm-Horsfall protein identified using a mass spectrometry experiment may be indicative of a particular renal disease. As such, the presence of such urological disease marker may be labelled as “familial juvenile hyperuricemic nephropathy.” Computing device 104 may train disease marker classifier 124 using disease marker training data 128. Disease marker training data 128 may be received and/or collected from experts or from users that may have may have been diagnosed with a urological disorder with particular disease markers. Disease marker training data 128 may be received as a function of determinations of urological disorders based on disease markers, urological disease metrics, and/or measurable values. Disease marker training data 128 set may be received and/or otherwise developed during one or more past iterations of the previous disease marker training data vectors. Disease marker training data 128 may be received from one or more remote devices that at least correlate disease markers related to urological disorders to urological disorder labels, where a remote device is an external device to computing device 104. “Training data,” as used herein, is data containing correlations that a machine-learning process may use to model relationships between two or more categories of data elements. For instance, and without limitation, training data may include a plurality of data entries, each entry representing a set of data elements that were recorded, received, and/or generated together; data elements may be correlated by shared existence in a given data entry, by proximity in a given data entry, or the like. Multiple data entries in training data may evince one or more trends in correlations between categories of data elements; for instance, and without limitation, a higher value of a first data element belonging to a first category of data element may tend to correlate to a higher value of a second data element belonging to a second category of data element, indicating a possible proportional or other mathematical relationship linking values belonging to the two categories. Multiple categories of data elements may be related in disease marker training data 128 according to various correlations; correlations may indicate causative and/or predictive links between categories of data elements, which may be modeled as relationships such as mathematical relationships by machine learning processes as described in further detail below. Disease marker training data 128 may be formatted and/or organized by categories of data elements, for instance by associating data elements with one or more descriptors corresponding to categories of data elements. As a non-limiting example, disease marker training data 128 may include data entered in standardized forms by persons or processes, such that entry of a given data element in a given field in a form may be mapped to one or more descriptors of categories. Elements in disease marker training data 128 may be linked to descriptors of categories by tags, tokens, or other data elements; for instance, and without limitation, disease marker training data 128 may be provided in fixed-length formats, formats linking positions of data to categories such as comma-separated value (CSV) formats and/or self-describing formats such as extensible markup language (XML), enabling processes or devices to detect categories of data.

Alternatively or additionally, and with continued reference to FIG. 1, disease marker training data 128 may include one or more elements that are not categorized; that is, disease marker training data 128 may not be formatted or contain descriptors for some elements of data. Machine learning algorithms and/or other processes may sort disease marker training data 128 according to one or more categorizations using, for instance, natural language processing algorithms, tokenization, detection of correlated values in raw data and the like; categories may be generated using correlation and/or other processing algorithms. As a non-limiting example, in a corpus of text, phrases making up a number “n” of compound words, such as nouns modified by other nouns, may be identified according to a statistically significant prevalence of n-grams containing such words in a particular order; such an n-gram may be categorized as an element of language such as a “word” to be tracked similarly to single words, generating a new category as a result of statistical analysis. Similarly, in a data entry including some textual data, a person's name may be identified by reference to a list, dictionary, or other compendium of terms, permitting ad-hoc categorization by machine learning algorithms, and/or automated association of data in the data entry with descriptors or into a given format. The ability to categorize data entries automatedly may enable the same disease marker training data 128 to be made applicable for two or more distinct machine learning algorithms as described in further detail below. Disease marker training data 128 used by computing device 104 may correlate any input data as described in this disclosure to any output data as described in this disclosure. Training data may contain entries, each of which correlates a machine learning process input to a machine learning process output, for instance without limitation, one or more elements of biological extraction data to a taste index. Training data may be obtained from previous iterations of machine-learning processes, user inputs, and/or expert inputs. Computing device 104 may train disease marker classifier 124 using disease marker training data 128. A description on machine learning and the use of classifiers follows below.

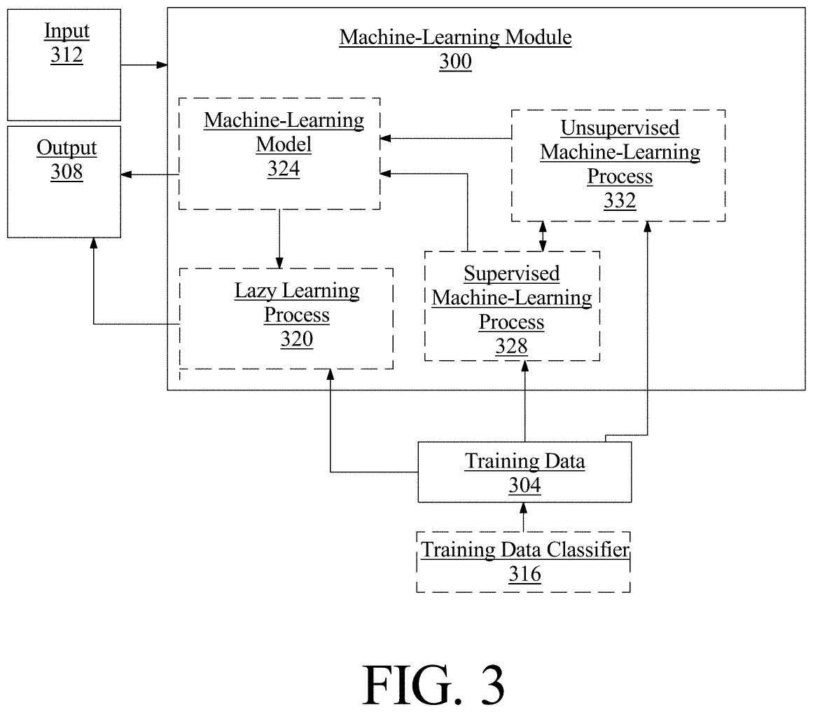

Referring now to FIG. 3, an exemplary embodiment of a machine-learning module 300 that may perform one or more machine-learning processes as described in this disclosure is illustrated. Machine-learning module may perform determinations, classification, and/or analysis steps, methods, processes, or the like as described in this disclosure using machine learning processes. A “machine learning process,” as used in this disclosure, is a process that automatedly uses training data 304 to generate an algorithm that will be performed by a computing device/module to produce outputs 308 given data provided as inputs 312; this is in contrast to a non-machine learning software program where the commands to be executed are determined in advance by a user and written in a programming language.

Still referring to FIG. 3, “training data,” as used herein, is data containing correlations that a machine-learning process may use to model relationships between two or more categories of data elements. For instance, and without limitation, training data 304 may include a plurality of data entries, each entry representing a set of data elements that were recorded, received, and/or generated together; data elements may be correlated by shared existence in a given data entry, by proximity in a given data entry, or the like. Multiple data entries in training data 304 may evince one or more trends in correlations between categories of data elements; for instance, and without limitation, a higher value of a first data element belonging to a first category of data element may tend to correlate to a higher value of a second data element belonging to a second category of data element, indicating a possible proportional or other mathematical relationship linking values belonging to the two categories. Multiple categories of data elements may be related in training data 304 according to various correlations; correlations may indicate causative and/or predictive links between categories of data elements, which may be modeled as relationships such as mathematical relationships by machine-learning processes as described in further detail below. Training data 304 may be formatted and/or organized by categories of data elements, for instance by associating data elements with one or more descriptors corresponding to categories of data elements. As a non-limiting example, training data 304 may include data entered in standardized forms by persons or processes, such that entry of a given data element in a given field in a form may be mapped to one or more descriptors of categories. Elements in training data 304 may be linked to descriptors of categories by tags, tokens, or other data elements; for instance, and without limitation, training data 304 may be provided in fixed-length formats, formats linking positions of data to categories such as comma-separated value (CSV) formats and/or self-describing formats such as extensible markup language (XML), JavaScript Object Notation (JSON), or the like, enabling processes or devices to detect categories of data.

Alternatively or additionally, and continuing to refer to FIG. 3, training data 304 may include one or more elements that are not categorized; that is, training data 304 may not be formatted or contain descriptors for some elements of data. Machine-learning algorithms and/or other processes may sort training data 304 according to one or more categorizations using, for instance, natural language processing algorithms, tokenization, detection of correlated values in raw data and the like; categories may be generated using correlation and/or other processing algorithms. As a non-limiting example, in a corpus of text, phrases making up a number “n” of compound words, such as nouns modified by other nouns, may be identified according to a statistically significant prevalence of n-grams containing such words in a particular order; such an n-gram may be categorized as an element of language such as a “word” to be tracked similarly to single words, generating a new category as a result of statistical analysis. Similarly, in a data entry including some textual data, a person's name may be identified by reference to a list, dictionary, or other compendium of terms, permitting ad-hoc categorization by machine-learning algorithms, and/or automated association of data in the data entry with descriptors or into a given format. The ability to categorize data entries automatedly may enable the same training data 304 to be made applicable for two or more distinct machine-learning algorithms as described in further detail below. Training data 304 used by machine-learning module 300 may correlate any input data as described in this disclosure to any output data as described in this disclosure. As a non-limiting illustrative example, disease markers for a urological disorder may serve as inputs, outputting other potential health disorders that a may use the same disease markers.

Further referring to FIG. 3, training data may be filtered, sorted, and/or selected using one or more supervised and/or unsupervised machine-learning processes and/or models as described in further detail below; such models may include without limitation a training data classifier 316. Training data classifier 316 may include a “classifier,” which as used in this disclosure is a machine-learning model as defined below, such as a mathematical model, neural net, or program generated by a machine learning algorithm known as a “classification algorithm,” as described in further detail below, that sorts inputs into categories or bins of data, outputting the categories or bins of data and/or labels associated therewith. A classifier may be configured to output at least a datum that labels or otherwise identifies a set of data that are clustered together, found to be close under a distance metric as described below, or the like. Machine-learning module 300 may generate a classifier using a classification algorithm, defined as a process whereby a computing device and/or any module and/or component operating thereon derives a classifier from training data 304. Classification may be performed using, without limitation, linear classifiers such as without limitation logistic regression and/or naive Bayes classifiers, nearest neighbor classifiers such as k-nearest neighbors classifiers, support vector machines, least squares support vector machines, fisher's linear discriminant, quadratic classifiers, decision trees, boosted trees, random forest classifiers, learning vector quantization, and/or neural network-based classifiers. As a non-limiting example, training data classifier 316 may classify elements of training data to classify a urological disorder into categories such as a target organ; a urological disease related to another disease state, and the like.

Still referring to FIG. 3, machine-learning module 300 may be configured to perform a lazy-learning process 320 and/or protocol, which may alternatively be referred to as a “lazy loading” or “call-when-needed” process and/or protocol, may be a process whereby machine learning is conducted upon receipt of an input to be converted to an output, by combining the input and training set to derive the algorithm to be used to produce the output on demand. For instance, an initial set of simulations may be performed to cover an initial heuristic and/or “first guess” at an output and/or relationship. As a non-limiting example, an initial heuristic may include a ranking of associations between inputs and elements of training data 304. Heuristic may include selecting some number of highest-ranking associations and/or training data 304 elements. Lazy learning may implement any suitable lazy learning algorithm, including without limitation a K-nearest neighbors algorithm, a lazy naïve Bayes algorithm, or the like; persons skilled in the art, upon reviewing the entirety of this disclosure, will be aware of various lazy-learning algorithms that may be applied to generate outputs as described in this disclosure, including without limitation lazy learning applications of machine-learning algorithms as described in further detail below.

Alternatively or additionally, and with continued reference to FIG. 3, machine-learning processes as described in this disclosure may be used to generate machine-learning model 324. A “machine-learning model,” as used in this disclosure, is a mathematical and/or algorithmic representation of a relationship between inputs and outputs, as generated using any machine-learning process including without limitation any process as described above and stored in memory; an input is submitted to a machine-learning model 324 once created, which generates an output based on the relationship that was derived. For instance, and without limitation, a linear regression model, generated using a linear regression algorithm, may compute a linear combination of input data using coefficients derived during machine-learning processes to calculate an output datum. As a further non-limiting example, a machine-learning model 324 may be generated by creating an artificial neural network, such as a convolutional neural network comprising an input layer of nodes, one or more intermediate layers, and an output layer of nodes. Connections between nodes may be created via the process of “training” the network, in which elements from a training data 304 set are applied to the input nodes, a suitable training algorithm (such as Levenberg-Marquardt, conjugate gradient, simulated annealing, or other algorithms) is then used to adjust the connections and weights between nodes in adjacent layers of the neural network to produce the desired values at the output nodes. This process is sometimes referred to as deep learning.

Still referring to FIG. 3, machine-learning algorithms may include at least a supervised machine-learning process 328. At least a supervised machine-learning process 328, as defined herein, include algorithms that receive a training set relating a number of inputs to a number of outputs, and seek to find one or more mathematical relations relating inputs to outputs, where each of the one or more mathematical relations is optimal according to some criterion specified to the algorithm using some scoring function. For instance, a supervised learning algorithm may include a disease marker such urinary NGAL, CyC, KIM-1, and hepatocyte growth factor as described above as inputs, with at acute kidney injury as outputs of a urological disorder, and a scoring function representing a desired form of relationship to be detected between inputs and outputs; scoring function may, for instance, seek to maximize the probability that a given input and/or combination of elements inputs is associated with a given output to minimize the probability that a given input is not associated with a given output. Scoring function may be expressed as a risk function representing an “expected loss” of an algorithm relating inputs to outputs, where loss is computed as an error function representing a degree to which a prediction generated by the relation is incorrect when compared to a given input-output pair provided in training data 304. Persons skilled in the art, upon reviewing the entirety of this disclosure, will be aware of various possible variations of at least a supervised machine-learning process 328 that may be used to determine relation between inputs and outputs. Supervised machine-learning processes may include classification algorithms as defined above.

Further referring to FIG. 3, machine learning processes may include at least an unsupervised machine-learning processes 332. An unsupervised machine-learning process, as used herein, is a process that derives inferences in datasets without regard to labels; as a result, an unsupervised machine-learning process may be free to discover any structure, relationship, and/or correlation provided in the data. Unsupervised processes may not require a response variable; unsupervised processes may be used to find interesting patterns and/or inferences between variables, to determine a degree of correlation between two or more variables, or the like.

Still referring to FIG. 3, machine-learning module 300 may be designed and configured to create a machine-learning model 324 using techniques for development of linear regression models. Linear regression models may include ordinary least squares regression, which aims to minimize the square of the difference between predicted outcomes and actual outcomes according to an appropriate norm for measuring such a difference (e.g. a vector-space distance norm); coefficients of the resulting linear equation may be modified to improve minimization. Linear regression models may include ridge regression methods, where the function to be minimized includes the least-squares function plus term multiplying the square of each coefficient by a scalar amount to penalize large coefficients. Linear regression models may include least absolute shrinkage and selection operator (LASSO) models, in which ridge regression is combined with multiplying the least-squares term by a factor of 1 divided by double the number of samples. Linear regression models may include a multi-task lasso model wherein the norm applied in the least-squares term of the lasso model is the Frobenius norm amounting to the square root of the sum of squares of all terms. Linear regression models may include the elastic net model, a multi-task elastic net model, a least angle regression model, a LARS lasso model, an orthogonal matching pursuit model, a Bayesian regression model, a logistic regression model, a stochastic gradient descent model, a perceptron model, a passive aggressive algorithm, a robustness regression model, a Huber regression model, or any other suitable model that may occur to persons skilled in the art upon reviewing the entirety of this disclosure. Linear regression models may be generalized in an embodiment to polynomial regression models, whereby a polynomial equation (e.g. a quadratic, cubic or higher-order equation) providing a best predicted output/actual output fit is sought; similar methods to those described above may be applied to minimize error functions, as will be apparent to persons skilled in the art upon reviewing the entirety of this disclosure.

Continuing to refer to FIG. 3, machine-learning algorithms may include, without limitation, linear discriminant analysis. Machine-learning algorithm may include quadratic discriminate analysis. Machine-learning algorithms may include kernel ridge regression. Machine-learning algorithms may include support vector machines, including without limitation support vector classification-based regression processes. Machine-learning algorithms may include stochastic gradient descent algorithms, including classification and regression algorithms based on stochastic gradient descent. Machine-learning algorithms may include nearest neighbors algorithms. Machine-learning algorithms may include Gaussian processes such as Gaussian Process Regression. Machine-learning algorithms may include cross-decomposition algorithms, including partial least squares and/or canonical correlation analysis. Machine-learning algorithms may include naïve Bayes methods. Machine-learning algorithms may include algorithms based on decision trees, such as decision tree classification or regression algorithms. Machine-learning algorithms may include ensemble methods such as bagging meta-estimator, forest of randomized tress, AdaBoost, gradient tree boosting, and/or voting classifier methods. Machine-learning algorithms may include neural net algorithms, including convolutional neural net processes.

With continued reference to FIG. 3, a “classifier,” as used in this disclosure, is a machine-learning model, such as a mathematical model, neural net, or program generated by a machine learning algorithm known as a “classification algorithm,” as described in further detail below, that sorts inputs into categories or bins of data, outputting the categories or bins of data and/or labels associated therewith. A classifier may be configured to output at least a datum that labels or otherwise identifies a set of data that are clustered together, found to be close under a distance metric as described below, or the like. Computing device 104 and/or another device may generate a classifier using a classification algorithm, defined as a processes whereby a computing device 104 derives a classifier from training data. Classification may be performed using, without limitation, linear classifiers such as without limitation logistic regression and/or naive Bayes classifiers, nearest neighbor classifiers such as k-nearest neighbors classifiers, support vector machines, least squares support vector machines, fisher's linear discriminant, quadratic classifiers, decision trees, boosted trees, random forest classifiers, learning vector quantization, and/or neural network-based classifiers.

Still referring to FIG. 3, computing device 104 may be configured to generate a classifier using a Naïve Bayes classification algorithm. Naïve Bayes classification algorithm generates classifiers by assigning class labels to problem instances, represented as vectors of element values. Class labels are drawn from a finite set. Naïve Bayes classification algorithm may include generating a family of algorithms that assume that the value of a particular element is independent of the value of any other element, given a class variable. Naïve Bayes classification algorithm may be based on Bayes Theorem expressed as P(A/B)═P(B/A) P(A)÷P(B), where P(A/B) is the probability of hypothesis A given data B also known as posterior probability; P(B/A) is the probability of data B given that the hypothesis A was true; P(A) is the probability of hypothesis A being true regardless of data also known as prior probability of A; and P(B) is the probability of the data regardless of the hypothesis. A naïve Bayes algorithm may be generated by first transforming training data into a frequency table. Computing device 104 may then calculate a likelihood table by calculating probabilities of different data entries and classification labels. Computing device 104 may utilize a naïve Bayes equation to calculate a posterior probability for each class. A class containing the highest posterior probability is the outcome of prediction. Naïve Bayes classification algorithm may include a gaussian model that follows a normal distribution. Naïve Bayes classification algorithm may include a multinomial model that is used for discrete counts. Naïve Bayes classification algorithm may include a Bernoulli model that may be utilized when vectors are binary.

With continued reference to FIG. 3, computing device 104 may be configured to generate a classifier using a K-nearest neighbors (KNN) algorithm. A “K-nearest neighbors algorithm” as used in this disclosure, includes a classification method that utilizes feature similarity to analyze how closely out-of-sample-features resemble training data to classify input data to one or more clusters and/or categories of features as represented in training data; this may be performed by representing both training data and input data in vector forms, and using one or more measures of vector similarity to identify classifications within training data, and to determine a classification of input data. K-nearest neighbors algorithm may include specifying a K-value, or a number directing the classifier to select the k most similar entries training data to a given sample, determining the most common classifier of the entries in the database, and classifying the known sample; this may be performed recursively and/or iteratively to generate a classifier that may be used to classify input data as further samples. For instance, an initial set of samples may be performed to cover an initial heuristic and/or “first guess” at an output and/or relationship, which may be seeded, without limitation, using expert input received according to any process as described herein. As a non-limiting example, an initial heuristic may include a ranking of associations between inputs and elements of training data. Heuristic may include selecting some number of highest-ranking associations and/or training data elements.

With continued reference to FIG. 3, generating k-nearest neighbors algorithm may generate a first vector output containing a data entry cluster, generating a second vector output containing an input data, and calculate the distance between the first vector output and the second vector output using any suitable norm such as cosine similarity, Euclidean distance measurement, or the like. Each vector output may be represented, without limitation, as an n-tuple of values, where n is at least two values. Each value of n-tuple of values may represent a measurement or other quantitative value associated with a given category of data, or attribute, examples of which are provided in further detail below; a vector may be represented, without limitation, in n-dimensional space using an axis per category of value represented in n-tuple of values, such that a vector has a geometric direction characterizing the relative quantities of attributes in the n-tuple as compared to each other. Two vectors may be considered equivalent where their directions, and/or the relative quantities of values within each vector as compared to each other, are the same; thus, as a non-limiting example, a vector represented as [5, 10, 15] may be treated as equivalent, for purposes of this disclosure, as a vector represented as [1, 2, 3]. Vectors may be more similar where their directions are more similar, and more different where their directions are more divergent; however, vector similarity may alternatively or additionally be determined using averages of similarities between like attributes, or any other measure of similarity suitable for any n-tuple of values, or aggregation of numerical similarity measures for the purposes of loss functions as described in further detail below. Any vectors as described herein may be scaled, such that each vector represents each attribute along an equivalent scale of values. Each vector may be “normalized,” or divided by a “length” attribute, such as a length attribute l as derived using a Pythagorean norm: l=Σᵢ₌₀ⁿaᵢ², where aᵢ is attribute number i of the vector. Scaling and/or normalization may function to make vector comparison independent of absolute quantities of attributes, while preserving any dependency on similarity of attributes; this may, for instance, be advantageous where cases represented in training data are represented by different quantities of samples, which may result in proportionally equivalent vectors with divergent values.

Alternatively or additionally, still with reference to FIG. 3, an exemplary embodiment of the generation of, for instance, a disease predictor score implementing a machine learning process is described. The machine-learning process may be implemented, without any limitations, as described earlier in this disclosure. A “disease predictor score,” as used in this disclosure, is a numerical value indicating a likelihood that at least one disease marker is indicative of a specific urological disorder. For instance, physiological data 116 which may contain a disease marker such as BTA may receive a higher disease predictor score when tagged with a label such as “Bladder Tumor” and a lower disease predictor score when tagged with a label such as “Overactive Bladder.” Although disease marker BTA may be associated with both a bladder tumor and overactive bladder, a higher disease predictor score would signify a higher probability that a positive BTA marker is related to a bladder tumor rather than an overactive bladder. Computing device 104 may receive disease predictor training data. Training data may be implemented, without limitation, as described earlier in this disclosure. Disease predictor training data may correlate disease marker related to urological disorders and urological disorder labels with a disease predictor score. Disease predictor training data may be received and/or collected from experts or from users that may have may have been diagnosed with a urological disorder with particular disease markers. Disease predictor training data may be received as a function of determinations of urological disorders based on disease markers, urological disease metrics, and/or measurable values and/or historical values which show a propensity of a user with at least one disease marker related to a urological disorder to have the urological disorder. Disease predictor training data set may be received from one or more past iterations of the previous predictor training data vectors. Disease predictor training data may be received from one or more remote devices that at least correlate disease markers related to urological disorders to urological disorder labels, wherein a remote device is an external device to computing device 104. Computing device 104 trains machine-learning process using disease predictor training data. Disease predictor score is generated, for each disease marker of a plurality of disease markers, based on machine-learning process and each respective disease marker of the plurality of disease markers. In an embodiment, computing device 104 is configured to identify a disease marker of the plurality of disease markers having a highest disease predictor score. Referring to the example above, computing device 104 the disease predictor score when BTA represents the disease marker and bladder tumor represents the label may be ranked higher relative to the disease predictor score of the same disease marker but with a label indicating an overactive bladder. As such, the disease predictor score for BTA as the disease marker labelled bladder tumor may receive the highest order. Computing device 104 may generate a nutritive plan based on the identification. Nutritive plans are described later in this disclosure.

Referring back to FIG. 1, computing device 104 may classify, using disease marker classifier 124, the at least disease marker 120 to urological disorder label 132. The use of classifiers may be implemented, without limitation, as described earlier in this disclosure. For instance, input 112 including physiological data 116 such as, but not limited to, urine, blood, semen, or the like. A disease marker such as neutrophil gelatinase-associated lipocalin (NGAL) and/or a chemokine such as chemokine 8 (CXCL8) may be present which may be labelled as an “urinary tract infection”; additionally, physiological data 116 may also contain abnormal levels of prostate-specific antigen which may be labelled “prostate cancer.” In another embodiment, disease marker classifier 124 may the at least disease marker 120 to a label related to a different disease state. For instance, physiological data 116 may contain disease markers RASSF1A and TIMP3. These markers may be associated with the label “urological disease.” These markers may also be associated and labelled for other types of disorders such as “schistosomiasis infection.”

With continued reference to FIG. 1, computing device 104 may generate nutritive plan 136 as a function of urological disorder label 132. As defined in this disclosure, an “nutritive plan” is a set of instructions for consumption of a plurality of nutritive compositions that, as used in this disclosure, may help relieve and/or prevent, for example, a urological disorder. “Nutritive compositions,” as used in this disclosure, may include any combination of ingredients that may be treated as a meal or a snack or any beverages or combination of beverages that may be consumed by a user. Nutritive plan 136 may include, for example, what type of nutritive compositions a user may want to consume based on the desire to relieve and/or prevent a urological disorder. Nutritive plan 136 may include what specific time of the day the user should consume the nutritive compositions. Nutritive plan 136 may include a list of nutritive compositions to avoid based on a urological disorder. Nutritive plan 136 may include a list of acceptable nutritive compositions substitutes in case a nutritive composition suggested to the user is not available. The nutritive plan may include a list of nutritional supplements that may relieve and/or prevent one or more urological disorder. The nutritive plan may include information as to how to safely take the supplements as well as information regarding any potential adverse effects. In an embodiment, a nutritive plan may address a plurality of disorders. For instance, a user may be suffering from a urological disorder in addition to another disorder such as hypertension. Computing device 104 may be configured to generate nutritive plan 136 which includes including consuming bananas daily to treat and/or prevent and/or improve the urological disorder and/or hypertension.

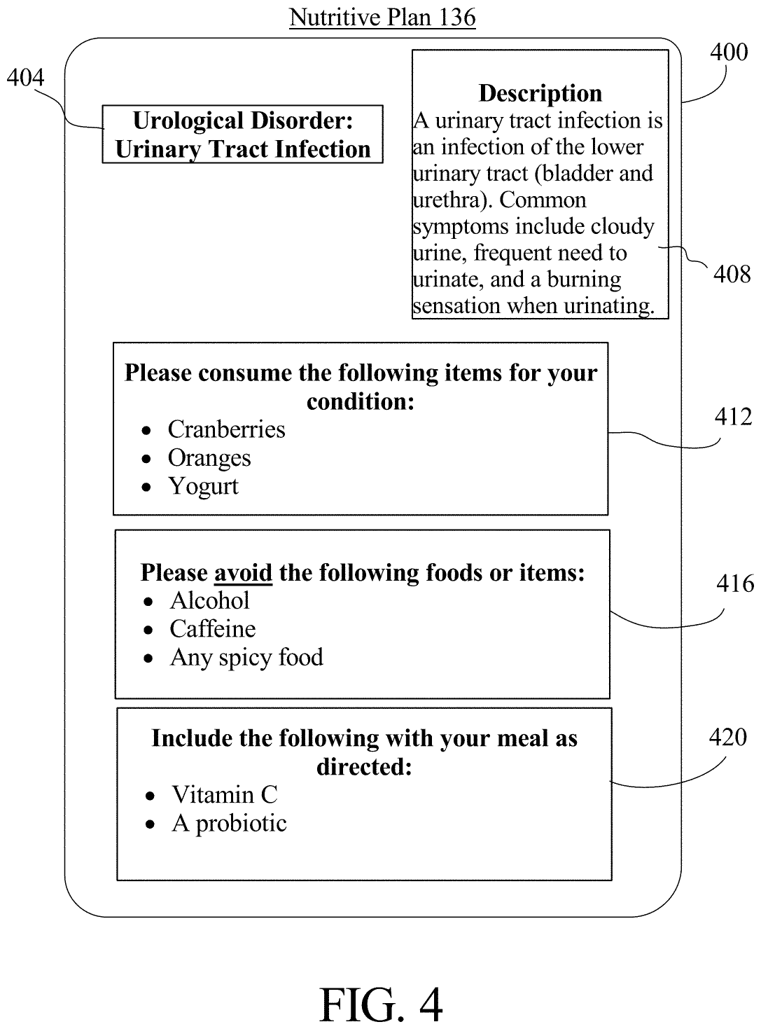

Referring now to FIG. 4, an exemplary embodiment of a nutritive plan is described. The nutritive plan may be outputted and displayed in a user device 400. For instance, nutritive plan 136 may be displayed in any GUI-based device, such as, but not limited to a mobile telephone, a tablet computer, a desktop computer, and the like. A user may be diagnosed with a urological disorder 404 such as, for example, a urinary tract infection. Nutritive plan 136 may include description section 408. Description section 408 may describe urological disorder 404. Nutritive plan 136 may include nutritive compositions 412 for the user to consume to relieve and/or prevent a urinary tract infection. Nutritive plan 136 may include adverse foods 416. Adverse foods 416 may include foods that a user should avoid as the foods listed may aggravate the urinary tract infection. Nutrients 420 may include a list if supplements that may relieve and/or prevent the urinary tract infection. Nutrients 420 may include, but not limited to, vitamins, amino acids, minerals, and the like.

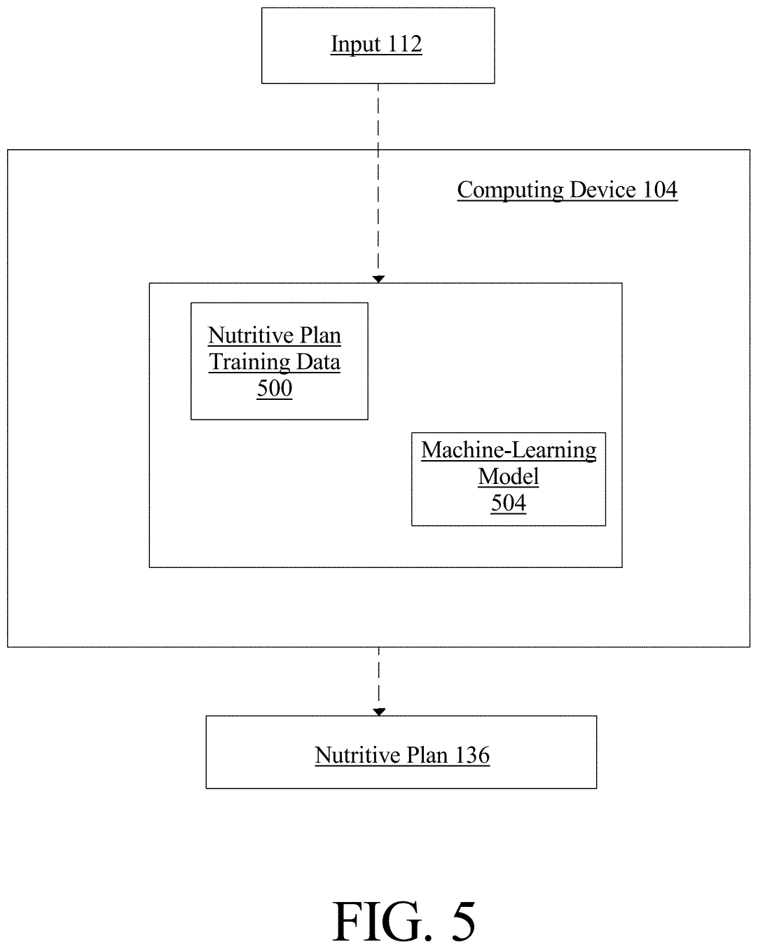

Now referring to FIG. 5, an exemplary embodiment of the generation of nutritive plan 136 implementing a machine learning process is described. Computing device 104 is configured to receive input 112. Computing device 104 may receive nutritive plan training data 500. Nutritive plan training data 500 may correlate nutritive plans with historical ameliorative and/or preventive effects on urological disorders. For example, nutritive plan training data 500 would include those nutritive plans that have relieved or prevented symptoms and/or effects of a urological disorder for past users. Nutritive plan training data 500 may be received and/or collected by experts or collected from users that may have received and used n nutritive plan to treat and/or prevent and/or improve a urological disorder. Nutritive plan training data 500 may be received as a function of user-entered valuations of nutritive plans, nutritive plan metrics, and/or measurable values. Nutritive plan training data 500 may be received from one or more past iterations of the previous nutritive plan vectors. Nutritive plan training data 500 may be received from one or more remote devices that at least correlate a nutritive plan element and urological disorder metric to a measurable value, wherein a remote device is an external device to computing device 104. A machine-learning process 504, which may include any machine-learning process as described in this disclosure may be trained and/or used to train models using nutritive plan training data 500. Nutritive plan 136 is outputted as a function of the urological disorder and machine-learning process 504. The machine-learning process may be implemented, without any limitations, as described earlier in this disclosure. In another embodiment, generating nutritive plan 136 may include outputting a message independent of a nutritive plan. For example, nutritive plan training data 500 may not contain values for a particular urological disorder. As a result, nutritive plan 136 may not be a suitable nutritive plan to treat and/or prevent and/or improve the particular urological disorder. As such, a message may be outputted indicating the unavailability of a suitable nutritive plan. The message may be outputted directly to a user device, a web page, an email message, and the like. An example of a message may include, “No nutrition suggestions are available for this urological disorder.”

Referring back to FIG. 1, in an embodiment, computing device 104 may be configured to receive a second input. The second input may include any of the inputs, without any limitations, as described for input 112. For example, a second input may correspond to a second urine taken after commencing implementation of nutritive plan 136. A medical professional may want to retest a user to check for changes in the presence or absence of disease marker 120. Computing device 104 may reclassify the at least one disease marker from the second input to a urological disorder label. Classification of the at least one disease marker from the second input to a urological disorder label may be implemented, without limitations, as described earlier in this disclosure. Computing device 104 may update nutritive plan 136 as a function of the second input. As a non-limiting example, disease marker 120 may remain present after implementing nutritive plan 136. Nutritive plan 136 generated to treat a urinary tract infection which may include, but not limited to a nutritive plan that includes green vegetables, carrots, and/or cruciferous vegetables may not reduce the presence of serum antibody immunoglobulin (Ig) G, IgM, and IgA which may indicate no improvement in the treatment of a urinary tract infection. Nutritive plan 136 may be updated to, for example, suggest the reduction or removal of carbonated beverages, alcohol, artificial sweeteners, and caffeine from a user's diet.

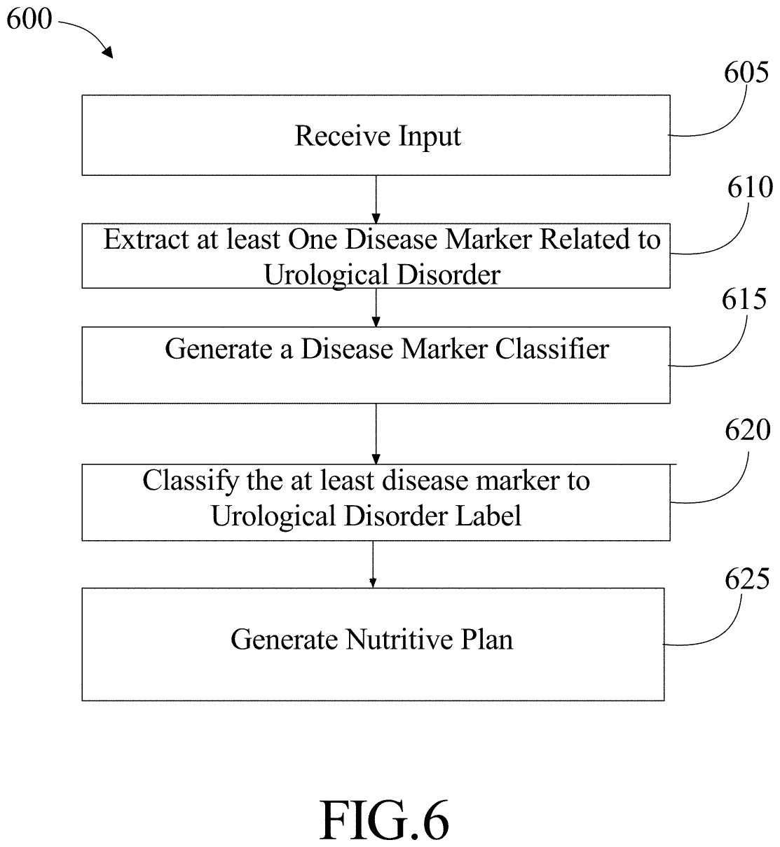

Referring now to FIG. 6, an exemplary method 600 for generating a nutritive plan to manage a urological disorder is described. At step 605, computing device receives an input. The input may include physiological data. This step may be implemented, without any limitations, as described in FIGS. 1-5. In an embodiment, the physiological data may include the results of a prostate-specific antigen test.

With continued reference to FIG. 6, at step 610, computing device may extract at least one disease marker related to at least one urological disorder. This step may be implemented, without any limitations, as described in FIGS. 1-5. In an embodiment, the at least one disease marker may include a diagnostic disease marker.

Still with reference to FIG. 6, at step 615, computing device may generate a disease marker classifier. Generating the disease marker classifier includes receiving disease marker training data. Disease marker training data correlates disease markers related to urological disorders to urological disorder labels. Computing device trains the disease marker classifier using the disease marker training data. This step may be implemented, without any limitation, as described in FIGS. 1-5.

Still referencing FIG. 6, at step 620, computing device may classify, using the disease marker classifier, the at least disease marker to a urological disorder label. This step may be implemented, without any limitation, as described in FIGS. 1-5.

With continued reference to FIG. 6, at step 625, computing device may generate a nutritive plan as a function of the urological disorder label. This step may be implemented, without any limitation, as described in FIGS. 1-5. In an embodiment, the nutritive plan may address a plurality of disorders. Computing device may output the nutritive plan to a user device.

Alternatively or additionally, with reference to FIG. 6, computing device receives predictor training data. The predictor training data correlates disease markers related to urological disorders and urological disorder labels with a disease predictor score. A machine-learning process is trained using the predictor training data. A disease predictor score is generated based in the machine-learning process and the at least one disease marker. In an embodiment, the disease predictor score is ordered in descending order, where the disease predictor score with a highest score receives a highest order. Computing device generates the nutritive plan as a function of the highest order. The limitations above may be implemented, without any limitations, as described earlier in the disclosure.

Alternatively or additionally, and still referencing FIG. 6, computing device receives nutritive plan training data. Nutritive plan training data may correlate nutritive plans to nutritive plans with historical ameliorative effect on urological disorders. Using the nutritive plan training data, a machine-learning process is trained. A nutritive plan is outputted as a function of the urological disorder and the machine-learning process. The limitations above may be implemented, without any limitations, as described earlier in the disclosure. In an embodiment, outputting the nutritive plan may include outputting a message independent of a presence of the nutritive plan. For example, nutritive plan training data 500 may not contain values for a particular urological disorder. As a result, nutritive plan 136 may not be a suitable nutritive plan to treat and/or prevent and/or improve the particular urological disorder. As such, a message may be outputted indicating the unavailability of a suitable nutritive plan. The message may be outputted directly to a user device, a web page, an email message, and the like. An example of a message may include, “No nutrition suggestions are available for this urological disorder.”

Alternatively or additionally, and with continued reference to FIG. 6, computing device may receive a second input. Computing device may reclassify the at least one disease marker from the second input to the positive result of the urological disorder. The nutritive plan is updated as a function of the second input. The limitations above may be implemented, without any limitations, as described earlier in the disclosure.

It is to be noted that any one or more of the aspects and embodiments described herein may be conveniently implemented using one or more machines (e.g., one or more computing devices that are utilized as a user computing device for an electronic document, one or more server devices, such as a document server, etc.) programmed according to the teachings of the present specification, as will be apparent to those of ordinary skill in the computer art. Appropriate software coding can readily be prepared by skilled programmers based on the teachings of the present disclosure, as will be apparent to those of ordinary skill in the software art. Aspects and implementations discussed above employing software and/or software modules may also include appropriate hardware for assisting in the implementation of the machine executable instructions of the software and/or software module.

Such software may be a computer program product that employs a machine-readable storage medium. A machine-readable storage medium may be any medium that is capable of storing and/or encoding a sequence of instructions for execution by a machine (e.g., a computing device) and that causes the machine to perform any one of the methodologies and/or embodiments described herein. Examples of a machine-readable storage medium include, but are not limited to, a magnetic disk, an optical disc (e.g., CD, CD-R, DVD, DVD-R, etc.), a magneto-optical disk, a read-only memory “ROM” device, a random-access memory “RAM” device, a magnetic card, an optical card, a solid-state memory device, an EPROM, an EEPROM, and any combinations thereof. A machine-readable medium, as used herein, is intended to include a single medium as well as a collection of physically separate media, such as, for example, a collection of compact discs or one or more hard disk drives in combination with a computer memory. As used herein, a machine-readable storage medium does not include transitory forms of signal transmission.

Such software may also include information (e.g., data) carried as a data signal on a data carrier, such as a carrier wave. For example, machine-executable information may be included as a data-carrying signal embodied in a data carrier in which the signal encodes a sequence of instruction, or portion thereof, for execution by a machine (e.g., a computing device) and any related information (e.g., data structures and data) that causes the machine to perform any one of the methodologies and/or embodiments described herein.

Examples of a computing device include, but are not limited to, an electronic book reading device, a computer workstation, a terminal computer, a server computer, a handheld device (e.g., a tablet computer, a smartphone, etc.), a web appliance, a network router, a network switch, a network bridge, any machine capable of executing a sequence of instructions that specify an action to be taken by that machine, and any combinations thereof. In one example, a computing device may include and/or be included in a kiosk.

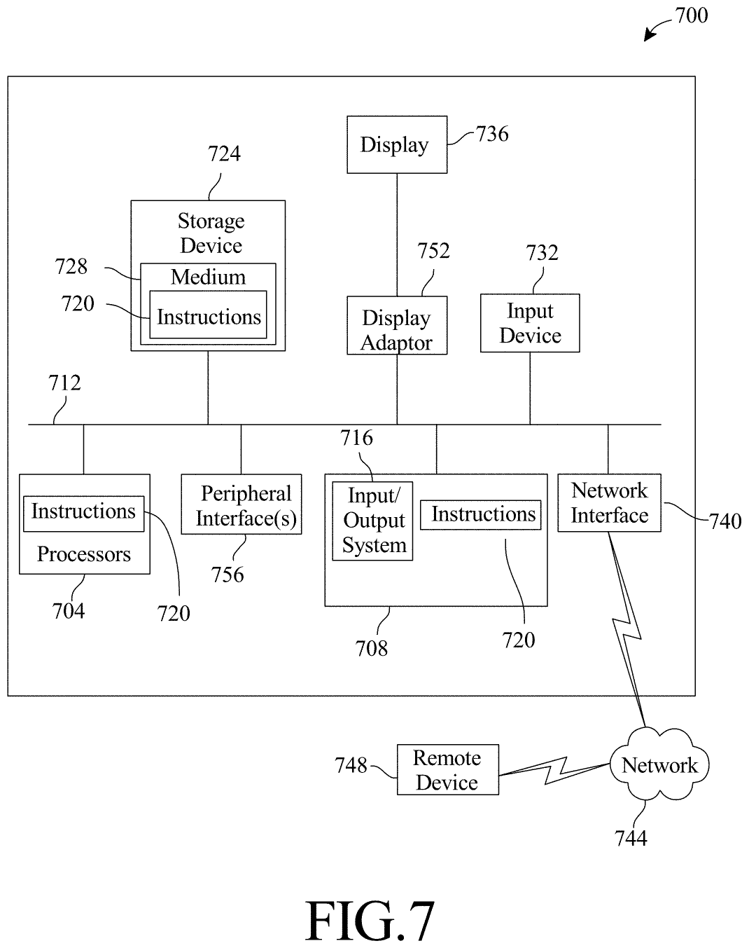

FIG. 7 shows a diagrammatic representation of one embodiment of a computing device in the exemplary form of a computer system 700 within which a set of instructions for causing a control system to perform any one or more of the aspects and/or methodologies of the present disclosure may be executed. It is also contemplated that multiple computing devices may be utilized to implement a specially configured set of instructions for causing one or more of the devices to perform any one or more of the aspects and/or methodologies of the present disclosure. Computer system 700 includes a processor 704 and a memory 708 that communicate with each other, and with other components, via a bus 712. Bus 712 may include any of several types of bus structures including, but not limited to, a memory bus, a memory controller, a peripheral bus, a local bus, and any combinations thereof, using any of a variety of bus architectures.

Processor 704 may include any suitable processor, such as without limitation a processor incorporating logical circuitry for performing arithmetic and logical operations, such as an arithmetic and logic unit (ALU), which may be regulated with a state machine and directed by operational inputs from memory and/or sensors; processor 704 may be organized according to Von Neumann and/or Harvard architecture as a non-limiting example. Processor 704 may include, incorporate, and/or be incorporated in, without limitation, a microcontroller, microprocessor, digital signal processor (DSP), Field Programmable Gate Array (FPGA), Complex Programmable Logic Device (CPLD), Graphical Processing Unit (GPU), general purpose GPU, Tensor Processing Unit (TPU), analog or mixed signal processor, Trusted Platform Module (TPM), a floating-point unit (FPU), and/or system on a chip (SoC)

Memory 708 may include various components (e.g., machine-readable media) including, but not limited to, a random-access memory component, a read only component, and any combinations thereof. In one example, a basic input/output system 716 (BIOS), including basic routines that help to transfer information between elements within computer system 700, such as during start-up, may be stored in memory 708. Memory 708 may also include (e.g., stored on one or more machine-readable media) instructions (e.g., software) 620 embodying any one or more of the aspects and/or methodologies of the present disclosure. In another example, memory 708 may further include any number of program modules including, but not limited to, an operating system, one or more application programs, other program modules, program data, and any combinations thereof.

Computer system 700 may also include a storage device 724. Examples of a storage device (e.g., storage device 724) include, but are not limited to, a hard disk drive, a magnetic disk drive, an optical disc drive in combination with an optical medium, a solid-state memory device, and any combinations thereof. Storage device 724 may be connected to bus 712 by an appropriate interface (not shown). Example interfaces include, but are not limited to, SCSI, advanced technology attachment (ATA), serial ATA, universal serial bus (USB), IEEE 1394 (FIREWIRE), and any combinations thereof. In one example, storage device 724 (or one or more components thereof) may be removably interfaced with computer system 700 (e.g., via an external port connector (not shown)). Particularly, storage device 724 and an associated machine-readable medium 728 may provide nonvolatile and/or volatile storage of machine-readable instructions, data structures, program modules, and/or other data for computer system 700. In one example, software 720 may reside, completely or partially, within machine-readable medium 728. In another example, software 720 may reside, completely or partially, within processor 704.

Computer system 700 may also include an input device 732. In one example, a user of computer system 700 may enter commands and/or other information into computer system 700 via input device 732. Examples of an input device 732 include, but are not limited to, an alpha-numeric input device (e.g., a keyboard), a pointing device, a joystick, a gamepad, an audio input device (e.g., a microphone, a voice response system, etc.), a cursor control device (e.g., a mouse), a touchpad, an optical scanner, a video capture device (e.g., a still camera, a video camera), a touchscreen, and any combinations thereof. Input device 732 may be interfaced to bus 712 via any of a variety of interfaces (not shown) including, but not limited to, a serial interface, a parallel interface, a game port, a USB interface, a FIREWIRE interface, a direct interface to bus 712, and any combinations thereof. Input device 732 may include a touch screen interface that may be a part of or separate from display device 736, discussed further below. Input device 732 may be utilized as a user selection device for selecting one or more graphical representations in a graphical interface as described above.

A user may also input commands and/or other information to computer system 700 via storage device 724 (e.g., a removable disk drive, a flash drive, etc.) and/or network interface device 740. A network interface device, such as network interface device 740, may be utilized for connecting computer system 700 to one or more of a variety of networks, such as network 744, and one or more remote devices 748 connected thereto. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus or other relatively small geographic space), a telephone network, a data network associated with a telephone/voice provider (e.g., a mobile communications provider data and/or voice network), a direct connection between two computing devices, and any combinations thereof. A network, such as network 744, may employ a wired and/or a wireless mode of communication. In general, any network topology may be used. Information (e.g., data, software 720, etc.) may be communicated to and/or from computer system 700 via network interface device 740.

Computer system 700 may further include a video display adapter 752 for communicating a displayable image to a display device, such as display device 736. Examples of a display device include, but are not limited to, a liquid crystal display (LCD), a cathode ray tube (CRT), a plasma display, a light emitting diode (LED) display, and any combinations thereof. Display adapter 752 and display device 736 may be utilized in combination with processor 704 to provide graphical representations of aspects of the present disclosure. In addition to a display device, computer system 700 may include one or more other peripheral output devices including, but not limited to, an audio speaker, a printer, and any combinations thereof. Such peripheral output devices may be connected to bus 712 via a peripheral interface 756. Examples of a peripheral interface include, but are not limited to, a serial port, a USB connection, a FIREWIRE connection, a parallel connection, and any combinations thereof.

The foregoing has been a detailed description of illustrative embodiments of the invention. Various modifications and additions can be made without departing from the spirit and scope of this invention. Features of each of the various embodiments described above may be combined with features of other described embodiments as appropriate in order to provide a multiplicity of feature combinations in associated new embodiments. Furthermore, while the foregoing describes a number of separate embodiments, what has been described herein is merely illustrative of the application of the principles of the present invention. Additionally, although particular methods herein may be illustrated and/or described as being performed in a specific order, the ordering is highly variable within ordinary skill to achieve methods and systems according to the present disclosure. Accordingly, this description is meant to be taken only by way of example, and not to otherwise limit the scope of this invention.

Exemplary embodiments have been disclosed above and illustrated in the accompanying drawings. It will be understood by those skilled in the art that various changes, omissions and additions may be made to that which is specifically disclosed herein without departing from the spirit and scope of the present invention.

What is claimed is:

1. A system for generating a nutritive plan to manage a urological disorder, the system comprising a computing device, wherein the computing device is configured to:
receive an input comprising physiological data;
extract at least one disease marker related to at least one urological disorder;
generate a disease marker classifier, wherein generating the disease marker classifier comprises:
receiving disease marker training data correlating disease markers related to urological disorders to a urological disorder label; and
training the disease marker classifier using the disease marker training data;
classify, using the disease marker classifier, the at least disease marker to a urological disorder label; and
generate a nutritive plan as a function of the urological disorder label.

2. The system of claim 1, wherein the computing device is further configured to:
receive disease predictor training data, wherein the disease predictor training data correlates disease markers related to urological disorders and urological disorder labels with disease predictor scores;
train, using the disease predictor training data, a machine-learning process; and
generate, for each disease marker of a plurality of disease markers, a disease predictor score as a function of the machine-learning process and each respective disease marker of the plurality of disease markers.

3. The system of claim 2, wherein the computing device is further configured to:
identify a disease marker of the plurality of disease markers having a highest disease predictor score; and
generate the nutritive plan as a function of the identification.

4. The system of claim 1, wherein the physiological data includes results of a prostate-specific antigen test.

5. The system of claim 1, wherein the at least one disease marker comprises a diagnostic disease marker.

6. The system of claim 1, wherein generating the nutritive plan further comprises:
receiving nutritive plan training data, wherein the nutritive plan training data correlates nutritive plans to nutritive plans with a historical ameliorative or preventive effect on urological disorders;
training a machine-learning process using the nutritive plan training data; and
outputting the nutritive plan as a function of the urological disorder and the machine-learning process.

7. The system of claim 6, wherein outputting the nutritive plan further comprises outputting a message independent of a presence of the nutritive plan.

8. The system of claim 1, wherein the computing device is further configured to output the nutritive plan to a user device.

9. The system of claim 1, wherein the nutritive plan manages a plurality of disorders.

10. The system of claim 1, wherein the computing device is further configured to:
receive a second input;
reclassify the at least one disease marker from the second input to a urological disorder label; and
update the nutritive plan as a function of the second input.

11. A method for generating a nutritive plan to manage a urological disorder, the method comprising:
receiving, by a computing device, an input comprising physiological data;
extracting, by the computing device, at least one disease marker related to at least one urological disorder;
generating, by the computing device, a disease marker classifier, wherein generating the disease marker classifier comprises:
receiving disease marker training data correlating disease markers related to urological disorders to a urological disorder label; and
training the disease marker classifier using the disease marker training data;
classifying, by the computing device and using the disease marker classifier, the at least disease marker to a urological disorder label; and
generating a nutritive plan as a function of the urological disorder label.

12. The method of claim 11, further comprising:
receiving disease predictor training data, wherein the disease predictor training data correlates disease markers related to urological disorders and urological disorder labels with disease predictor scores;
training, using the disease predictor training data, a machine-learning process; and
generating, for each disease marker of a plurality of disease markers, a disease predictor score as a function of the machine-learning process and each respective disease marker of the plurality of disease markers.

13. The method of claim 12, further comprising:
identifying a disease marker of the plurality of disease markers having a highest disease predictor score; and
generating the nutritive plan as a function of the identification.

14. The method of claim 11, wherein the physiological data includes results of a prostate-specific antigen test.

15. The method of claim 11, wherein the at least one disease marker comprises a diagnostic disease marker.

16. The method of claim 11, wherein generating the nutritive plan further comprises:
receiving nutritive plan training data, wherein the nutritive plan training data correlates nutritive plans to nutritive plans with a historical ameliorative or preventive effect on urological disorders;
training a machine-learning process using the nutritive plan training data; and
outputting the nutritive plan as a function of the urological disorder and the machine-learning process.

17. The method of claim 16, wherein outputting the nutritive plan further comprises outputting a message independent of a presence of the nutritive plan.

18. The method of claim 11, further comprising outputting the nutritive plan to a user device.

19. The method of claim 11, wherein the nutritive plan manages a plurality of disorders.

20. The method of claim 11, further comprising:
receiving a second input;
reclassifying the at least one disease marker from the second input to a urological disorder label; and
updating the nutritive plan as a function of the second input.
